(19) United States
(12) Patent Application Publication
(10) Pub. No.: US 2025/0163113 A1
(43) Pub. Date: May 22, 2025

(54) PEPTIDES TARGETING THE INTERACTION BETWEEN KINDLIN-1 AND Beta-INTEGRIN

(71) Applicant: INSTITUT CURIE, PARIS (FR)

(72) Inventor: Keltouma DRIOUCH, Paris Cedex 05 (FR)

(21) Appl. No.: 18/840,554

(22) PCT Filed: Feb. 24, 2023
(86) PCT No.: PCT/EP2023/054731

§ 371 Date: Aug. 22, 2024

(30) Foreign Application Priority Data
Feb. 25, 2022 (EP) 22305214.3

(51) Int. Cl. C07K 14/47; A61K 38/00; A61P 35/04; C07K 7/06; C07K 7/08

(52) U.S. Cl. CPC C07K 14/47; A61P 35/04; C07K 7/06; C07K 7/08; A61K 38/00; C07K 2319/10; C07K 2319/31; C07K 2319/60

(57) ABSTRACT

The present invention concerns novel peptides targeting the interaction between Kindlin-1 and β-integrin, and pharmaceutical compositions comprising these peptides. The invention also relates to these peptides and compositions for use in a method for preventing and/or treated cancer in a subject.

The present invention concerns novel peptides targeting the interaction between Kindlin-1 and β-integrin, and pharmaceutical compositions comprising these peptides. The invention also relates to these peptides and compositions for use in a method for preventing and/or treating cancer in a subject.

Cancer is the second leading cause of death globally, and is responsible for an estimated 9.6 million deaths in 2018. Globally, about 1 in 6 deaths is due to cancer. The most common types of cancer in males are lung cancer, prostate cancer, colorectal cancer and stomach cancer. In females, the most common types are breast cancer, colorectal cancer, lung cancer and cervical cancer.

Altered expression levels of Kindlin-1 have been reported in a broad range of cancers. It was previously shown that, at a clinical level, Kindlin-1 expression is higher in the tumors than in the normal tissues in different cancer types (breast, lung, colon, bladder . . . ) and its up-regulation is associated with a worse prognosis. Kindlin-1 expression was found to increase cellular proliferation, migration and invasion.

Kindlin-1 is a focal adhesion protein involved in the activation of β-integrins and therefore participate in important cellular processes such as cell adhesion, proliferation or migration.

The inventors established a mouse breast cancer cell line expressing wild type or a mutant form of Kindlin-1 unable to bind to β-integrins (respectively 168FARN Kind1 and AAKind1). The expression of this mutant inhibited β-integrin activation and decreased cell motility and invasion in vitro. It also inhibited metastasis in vivo. The inventors thus developed peptides targeting the interaction between Kindlin-1 and β-integrin to recapitulate these anti-tumoral phenotypes. They developed a new therapeutic strategy based on selective pharmacological inhibitors of Kindlin-1 for the treatment of cancers overexpressing this protein.

### SUMMARY OF THE INVENTION

Thus, in a first aspect, the invention provides an isolated peptide comprising the amino acid sequence SEQ ID NO: 1:

(SEQ ID NO: 1)
SFLRM

wherein said isolated peptide comprises from 5 to 50 amino acids, and said isolated peptide does not consist of sequence PRRSFLRMP (SEQ ID NO:40).

Also provided is an isolated peptide comprising the amino acid sequence SEQ ID NO: 14:

(SEQ ID NO: 14)
QYHISKLSLSAETQDF

wherein said isolated peptide comprises from 5 to 50 amino acids.

In a further aspect, the invention provides a pharmaceutical composition comprising a peptide of the invention.

The invention also concerns a peptide comprising the amino acid sequence SEQ ID NO: 1 or SEQ ID NO:14 or a pharmaceutical composition comprising a peptide comprising the amino acid sequence SEQ ID NO: 1 or SEQ ID NO:14 for use in a method for treating cancer in a subject.

The invention also relates to a peptide comprising the amino acid sequence SEQ ID NO: 1 or SEQ ID NO:14 or a pharmaceutical composition comprising a peptide comprising the amino acid sequence SEQ ID NO: 1 or SEQ ID NO: 14 for use in a method for preventing and/or treating metastases in a subject having cancer.

The invention further relates to a polypeptide that comprises a peptide of the invention, wherein the polypeptide comprises no more than 50 consecutive amino acids of human Kindlin-1

### DESCRIPTION OF THE INVENTION

### Definitions

In the context of the invention, the term “peptide” refers to native peptides (either proteolysis products or synthetically synthesized peptides) and further to peptidomimetics, such as peptoids and semipeptoids which are peptide analogs, which may have, for example, modifications rendering the peptides more stable while in a body, or more immunogenic. Such modifications include, but are not limited to, cyclization, N-terminus modification, C-terminus modification, acylation, PEGylation, peptide bond modification, including, but not limited to, CH₂—NH, CH₂—S, CH₂—S═O, O═C—NH, CH₂—O, CH₂—CH₂, S═C—NH, CH═CH or CF═CH, backbone modification and residue modification.

By “peptide”, it is meant an amino acid sequence comprising from 5 to 50 amino acids (i.e. consisting of 5 to 50 amino acids).

As used herein, the term “amino acid” is understood to include the 20 naturally occurring amino acids i.e. alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine; amino acids harbouring the post-translational modifications which can be found in vivo such as hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

As used herein, the term “isolated peptide” refers to any peptide, irrespective of its method of synthesis, which is locationally distinct from the naturally occurring protein sequence of which it may form a part in nature.

### Peptide

As indicated above and shown in the examples below, the inventors designed two peptides able to inhibit the interaction between Kindlin-1 and β-integrins.

As used herein, the terms “inhibitor of the interaction” means preventing or reducing the direct or indirect association of one or more molecules, nucleic acids, peptide or proteins.

Accordingly, the present invention concerns an isolated peptide comprising the amino acid sequence SEQ ID NO: 1:
SFLRM (SEQ ID NO: 1) wherein said isolated peptide comprises from 5 to 50 amino acids, and said isolated peptide does not consist of sequence PRRSFLRMP (SEQ ID NO:40).

In some embodiments, the isolated peptide comprises or consists of a fragment of no more than 50 consecutive amino acids of human Kindlin-1 (SEQ ID NO: 41), preferably no more than 40, 35, 30, 25 or 20 consecutive amino acids of human Kindlin-1.

In some embodiments, the isolated peptide does not comprise sequence PRRSFLRMP (SEQ ID NO:40).

In a particular embodiment, said peptide further comprises at least one amino acid at the carboxyl-terminal end of sequence SEQ ID NO: 1. Preferably, the first amino acid directly after the carboxyl-terminal end of sequence SEQ ID NO: 1 is other than proline, and more preferably the first amino acid directly after the carboxyl-terminal end of sequence SEQ ID NO: 1 is a lysine.

In another particular embodiment, the peptide according to the invention comprises at least 7 consecutive amino acid sequence of SEQ ID NO: 2:

(SEQ ID NO: 2)
LNILSFLRMKNRNSASQVASSL.

In another particular embodiment, the peptide according to the invention has at least 75%, 80%, 85% or 90% of identity with the amino acid sequence SEQ ID NO: 2.

Amino acid sequence identity is defined as the percentage of amino acid residues in the sequence that are identical with the amino acid residues in the reference sequence, after aligning the sequences and introducing gaps, if necessary, to achieve the maximum percent sequence identity, and not considering any conservative substitutions as part of the sequence identity. Sequence identity may be determined over the full length of the analysed sequence, the full length of the reference sequence, or both. The percentage of identity for protein sequences may be calculated by performing a pairwise global alignment based on the Needleman-Wunsch alignment algorithm to find the optimum alignment (including gaps) of two sequences along their entire length, for instance using Needle, and using the BLOSUM62 matrix with a gap opening penalty of 10 and a gap extension penalty of 0.5.

In a preferred embodiment, the peptide according to the invention is selected from the group consisting of the peptides comprising or consisting of amino acid sequences:

```
  (SEQ ID NO: 3)
SFLRMKNRNSASQVASSL,
  (SEQ ID NO: 4)
SFLRMKNRNSASQVA,
  (SEQ ID NO: 5)
SFLRMKNRNSASQ,
  (SEQ ID NO: 6)
ILSFLRMKNRNS,
  (SEQ ID NO: 7)
LNILSFLRMKNR,
  (SEQ ID NO: 8)
SFLRMKNRNSA,
  (SEQ ID NO: 9)
SFLRMKNRNS,
  (SEQ ID NO: 10)
SFLRMKNRN,
  (SEQ ID NO: 11)
SFLRMKNR,
  (SEQ ID NO: 12)
SFLRMKN,
  (SEQ ID NO: 13)
SFLRMK,
and
(SEQ ID NO: 1)
SFLRM.
```

More preferably, the peptide according to the invention is the peptide comprising or consisting of amino acid sequence SFLRMKNRNSASQVASSL (SEQ ID NO: 3).

The present invention also concerns an isolated peptide comprising the amino acid sequence SEQ ID NO: 14:

(SEQ ID NO: 14)
QYHISKLSLSAETQDF

wherein said isolated peptide comprises from 5 to 50 amino acids.

Peptides according to the invention may further comprise a peptide tag, a cell penetrating peptide and/or a compound extending half-life of the peptide, at its amino-terminal or Carboxyl-terminal end, in particular at its Carboxyl-terminal end.

In some embodiments such a peptide tag, cell penetrating peptide and/or compound extending half-life of the peptide makes part of the peptide of the invention. In some embodiments such a peptide tag, cell penetrating peptide and/or compound extending half-life of the peptide makes part of a polypeptide that comprises the peptide of the invention. In some embodiments the polypeptide consists of 51 to 100 amino acids.

Any of a variety of art recognized peptide tags can be employed in the present invention. For example, suitable peptide tags include a: FLAG peptide, short FLAG peptide, His-6 peptide, Glutathione-S-Transferase (GST), Staphylococcal protein A, Streptococcal protein G, Calmodulin, Calmodulin binding peptides, Thioredoxin, β-galactosidase, Ubiquitin, Chloramphenicol cetyltransferasel S-peptide (Ribonuclease A, residues 1-20), Myosin heavy chain, DsbA, Biotin subunit, Avidin, Streptavidin, Sfrp-tag, c-Myc, Dihydrofolate reductase, CKS, Polyarginine, Polycysteine, Polyphenylalanine, lac Repressor, N-terminus of the growth hormone, Maltose binding protein, Galactose binding protein, Cyclomaltodextrin glucanotransferase, Callulose binding domain, Haemolysin A, ToE or ToLE, Protein kinase sites, BAI epitope, Btag, VP7 region of Bluetongue virus, Green Fluorescent Protein or any fluorochromes.

The peptide tag can include one or more specific protease cleavage sites.

Any of a variety of art recognized cell penetrating peptide (CPP) can be employed in the present invention. In some embodiments, the cell-penetrating peptide is a Transactivator of Transcription (TAT) cell penetrating sequence, a cell permeable peptide or a membranous penetrating sequence. The term “cell-penetrating peptides” are well known in the art and refers to cell permeable sequence or membranous penetrating 20 sequence such as penetratin, TAT mitochondrial penetrating sequence and compounds (Bechara and Sagan, 2013; Jones and Sayers, 2012; Khafagy el and Morishita, 2012; Malhi and Murthy, 2012). In some embodiments, the heterologous polypeptide is an internalization sequence derived either from the homeodomain of Drosophila Antennapedia/Penetratin (Antp) protein or a Transactivator of Transcription (TAT) cell 25 penetrating sequence. For example, suitable cell penetrating peptides according to the invention, include Transactivating transcriptional activator (TAT), Pep-1, Mut3DPT (of sequence SEQ ID NO: 17).

Preferably, the peptide or polypeptide according to the invention comprise Mut3DPT sequence at its amino-terminal end.

In a preferred embodiment, the isolated peptide according to the invention is selected from the group consisting of the peptides comprising or consisting of amino acid sequences:

```
  (SEQ ID NO: 15)
VKKKKIKAEIKISFLRMKNRNSASQVASSL,
  (SEQ ID NO: 20)
VKKKKIKAEIKIILSFLRMKNRNS,
  (SEQ ID NO: 21)
VKKKKIKAEIKILNILSFLRMKNR,
  (SEQ ID NO: 23)
VKKKKIKAEIKISFLRM,
  (SEQ ID NO: 24)
VKKKKIKAEIKISFLRMK,
  (SEQ ID NO: 25)
VKKKKIKAEIKISFLRMKN,
  (SEQ ID NO: 26)
VKKKKIKAEIKISFLRMKNR,
  (SEQ ID NO: 27)
VKKKKIKAEIKISFLRMKNRN,
  (SEQ ID NO: 28)
VKKKKIKAEIKISFLRMKNRNS,
  (SEQ ID NO: 29)
VKKKKIKAEIKISFLRMKNRNSA,
  (SEQ ID NO: 30)
VKKKKIKAEIKISFLRMKNRNSASQ,
  (SEQ ID NO: 31)
VKKKKIKAEIKISFLRMKNRNSASQVA,
and
  (SEQ ID NO: 16)
VKKKKIKAEIKIQYHISKLSLSAETQDF.
```

In a particular embodiment, the isolated peptide according to the invention comprises or consists in the peptide of the amino acid sequence VKKKKIKAEIKISFLRMKNRNSASQVASSL (SEQ ID NO: 15) or the peptide of amino acid sequence VKKKKIKAEIKIQYHISKLSLSAETQDF (SEQ ID NO: 16).

Any of a variety of art recognized compound extending half-life of the peptide can be employed in the present invention. In particular embodiments, the peptide of the invention is modified so that its stability, in particular in vivo, and/or its circulation time is increased, compared to non-modified peptides. Potential modifications that may be performed include PEGylation, acylation, biotinylation, acetylation, formylation, ubiquitination, amidation, enzyme labeling, or radiolabeling. For instance, varying degrees of PEGylation may be used to vary the half-life of the peptide, with increased PEGylation corresponding to increased half-life. Modifications may occur at any location on the peptide, including the peptide backbone, the amino acid side chains, and the amino or carboxy termini.

In another embodiment, the peptide of the invention may be modified by addition of the Fc domain of an antibody. The Fc domain of an antibody is a relatively constant region that is responsible for biological activity rather than antigen binding. A variety of therapeutic polypeptides has been created using the Fc domain to increase the half-life of the polypeptide. Attachment of an Fc domain to the peptide of the present invention is likely to increase the half-life of the peptide. The Fc domain may comprise portions of a digested, naturally occurring antibody, or it may be derived from a recombinant or humanized antibody.

In a further aspect, it is provided a polypeptide that comprises any of the peptides of the invention. The polypeptide comprises no more than 50 consecutive amino acids of human Kindlin-1, preferably no more than 40, 35, 30, 25 or 20 consecutive amino acids of human Kindlin-1. In some embodiments, the polypeptide consists of 51 to 100 amino acids.

In a preferred embodiment, the peptide or polypeptide according to the invention inhibits the interaction between Kindlin-1 and β-integrins, in particular with β1-integrin, and thereby reduces the activation of β-integrins, in particular β1-integrin.

### Pharmaceutical Composition

The present invention also concerns a pharmaceutical composition comprising a peptide as defined the section “Peptide” above, and optionally a pharmaceutically acceptable excipient.

The term “pharmaceutically acceptable” refers to properties and/or substances which are acceptable for administration to a subject from a pharmacological or toxicological point of view. Further “pharmaceutically acceptable” refers to factors such as formulation, stability, patient acceptance and bioavailability which will be known to a manufacturing pharmaceutical chemist from a physical/chemical point of view.

As used herein, “pharmaceutically acceptable excipient” refers to any substance in a pharmaceutical composition different from the active ingredient. Said excipients can be liquids, sterile, as for example water and oils, including those of origin in the petrol, animal, vegetable or synthetic, as peanut oil, soy oil, mineral oil, sesame oil, and similar, disintegrate, wetting agents, solubilizing agents, antioxidant, antimicrobial agents, isotonic agents, stabilizing agents or diluents.

The pharmaceutical compositions of the invention can be formulated for a parenteral, e.g., intravenous, intradermal, intracerebroventricular, subcutaneous, intramuscular, intraperitoneal, oral (e.g., buccal, inhalation, nasal and pulmonary spray), intradermal, transdermal (topical), transmucosal or intratumoral administration.

### Medical Indications

The inventors demonstrated that the peptides of the invention permit a strong reduction in the capacities of breast cancer cells to proliferate, migrate, degrade the extracellular matrix and invade, in vitro; and to generate lung metastasis in vivo. In addition, these peptides specifically induce cell death in EGFR and KRAS-dependent cancer cell lines of diverse epithelial tissue origins. Accordingly, peptides of the invention are particularly interesting to treat cancer.

The invention thus also concerns a peptide comprising the amino acid sequence SEQ ID NO: 1 or SEQ ID NO:14, or a pharmaceutical composition comprising a peptide comprising the amino acid sequences SEQ ID NO: 1 or SEQ ID NO:14, for use in a method for treating cancer in a subject.

The invention also concerns a peptide comprising amino acid sequences SEQ ID NO: 1 or SEQ ID NO:14, or a pharmaceutical composition comprising a peptide comprising the amino acid sequences SEQ ID NO: 1 or SEQ ID NO:14, for use in a method for preventing and/or treating metastases in a subject having cancer.

In a preferred embodiment, the peptide is as defined in the section “Peptide” above and the pharmaceutical composition as defined in the section “Pharmaceutical composition”.

The present invention also relates to a peptide as defined in the section “Peptide” above or a pharmaceutical composition as defined in the section “Pharmaceutical composition” above for use in a method for treating cancer in a subject.

The present invention also concerns the use of a peptide as defined in the section “Peptide” above or of a pharmaceutical composition as defined in the section “Pharmaceutical composition” above for the manufacture of a medicament intended to treat cancer.

The present invention also concerns a method for treating cancer in a subject, comprising administering to a subject in need thereof a therapeutically effective amount of a peptide as defined in the section “Peptide” above or a pharmaceutical composition as defined in the section “Pharmaceutical composition” above.

The present invention also relates to a peptide as defined in the section “Peptide” above or a pharmaceutical composition as defined in the section “Pharmaceutical composition” above for use in a method for preventing and/or treating metastases in a subject having cancer.

The present invention also concerns the use of a peptide as defined in the section “Peptide” above or of a pharmaceutical composition as defined in the section “Pharmaceutical composition” above for the manufacture of a medicament intended to prevent and/or treat metastases in a subject having cancer.

The present invention also concerns a method for preventing and/or treating metastases in a subject having cancer, comprising administering to a subject in need thereof a therapeutically effective amount of a peptide as defined in the section “Peptide” above or a pharmaceutical composition as defined in the section “Pharmaceutical composition” above.

By “cancer”, it meant herein a disease involving abnormal cell growth with the potential to invade or spread to other parts of the body. The latter process is called metastasizing and is a major cause of death from cancer. Neoplasm and malignant tumor are other common names for cancer. Lung, prostate, colorectal, stomach and liver cancer are the most common types of cancer in men, while breast, colorectal, lung, cervical and thyroid cancer are the most common among women.

In the context of the invention, the term “preventing” or “prevention” refers to the prophylactic treatment of a subject who is at risk of developing a condition resulting in a decrease in the probability that the subject will develop the condition or a delay in the development of the condition. In particular herein, the probability that the subject will develop metastases or new metastases is decrease and/or the development of metastases or new metastases is delayed.

“Therapy” or “treatment” or “treating” includes reducing, alleviating, inhibiting, or eliminating the causes of a disease or pathological conditions, as well as treatment intended to reduce, alleviate, inhibit or eliminate symptoms of said disease or pathological condition.

In particular herein, “treating cancer” includes reducing the tumor size, slowing the tumor growth and/or eliminating the tumor.

In particular herein, “treating metastases” includes reducing at least one metastases' size, slowing at least one metastases' growth and/or eliminating at least one metastase.

By a “therapeutically effective amount” of a peptide or a pharmaceutical composition of the invention is meant a sufficient amount of the peptide or composition to treat or prevent said disease or disorder, at a reasonable benefit/risk ratio applicable to any medical treatment. It will be understood, however, that the total daily usage of the peptide or composition of the present invention will be decided by the attending physician within the scope of sound medical judgment. The specific therapeutically effective dose level for any particular subject will depend upon a variety of factors including the disorder being treated and the severity of the disorder, activity of the specific peptides or compositions employed, the specific combinations employed, the age, body weight, general health, sex and diet of the subject, the time of administration, route of administration and rate of excretion of the specific peptides employed, the duration of the treatment, drugs used in combination or coincidental with the specific peptides employed, and like factors well known in the medical arts. For example, it is well within the skill of the art to start doses of the peptides at levels lower than those required to achieve the desired therapeutic effect and to gradually increase the dosage until the desired effect is achieved.

The form of the pharmaceutical compositions, the route of administration, the dosage and the regimen naturally depend upon the condition to be treated or prevented, the severity of the illness, the age, weight, and sex of the patient, etc.

As used herein the term “subject” refers to a mammalian such as a rodent, a feline, an equine, a bovine, an ovine, a canine or a primate, and is preferably a primate and more preferably a human. The subject has been diagnosed as having a cancer, and preferably a solid cancer, in particular an epithelial cancer.

The peptides and pharmaceutical compositions of the invention can be administered by any suitable route, in particular by parenteral, e.g., intravenous, intradermal, intracerebroventricular, subcutaneous, intramuscular, intraperitoneal, oral (e.g., buccal, inhalation, nasal and pulmonary spray), intradermal, transdermal (topical), transmucosal or intratumoral route.

In an embodiment, the cancer is breast, lung, colon, pancreas, bladder, or head and neck cancer, preferably the cancer is a triple negative breast cancer. In an embodiment, the cancer is breast, pancreas, bladder, or head and neck cancer. In an embodiment, the cancer is pancreas, bladder, or head and neck cancer.

In a particular embodiment, the cancer comprises cells with high Kindlin-1 expression level.

By “cancer cells”, it is meant cells extracted from a cancerous tumor of a patient. These cells can be alive or lysed. They can be processed (such as purification, fractionation, enzymatic processing, freezing etc. . . . ) prior to the measuring of the expression level of Kindlin-1.

By “Kindlin-1 expression level”, it is meant the level of expression of messenger RNA (mRNA) encoding Kindlin-1 and/or the level of expression of the Kindlin-1 protein.

Preferentially, the level of expression of Kindlin-1 protein is measured using a specific ligand of Kindlin-1, such as for example an antibody, preferably monoclonal antibody, a Fab fragment, an scFv or a nanobody, specific for Kindlin-1. The level of expression may then be measured by means of any method known to those skilled in the art, such as for example by means of a Western Blot or an ELISA test. The level of expression of Kindlin-1 protein may also be measured by mass analysis, such as mass spectrometry. Qualitative and quantitative mass spectrometric techniques are known and used in the art. A quantitative LC-MS/MS can also be used.

Preferentially, the level of expression of mRNA of the gene encoding Kindlin-1 is measured using a complementary nucleotide sequence of the mRNA of the gene encoding Kindlin-1 and specifically hybridizing with the mRNA of the gene encoding Kindlin-1 or, a fragment thereof hybridizing specifically with the mRNA of the gene encoding Kindlin-1, this sequence or this fragment comprising 5 to 50 nucleotides, preferentially 10 to 20 nucleotides, or using a pair of primers or a probe of 10 to 60 nucleotides, preferentially 15 to 30 nucleotides comprising said sequence or said fragment. The level of expression may then be measured by any means known to those skilled in the art, for example by means of quantitative RT-PCR.

Within the scope of the invention, the terms “hybridize” or “hybridization”, are well-known to those skilled in the art, refer to the binding of a nucleic acid sequence with a particular nucleotide sequence under suitable conditions, particularly under stringent conditions.

The term “stringent conditions”, as used herein, corresponds to conditions suitable for producing bond pairs between the nucleic acids having a defined level of complementarity, while being unsuitable for the formation of pairs between the bonding nucleic acids having a lower complementarity than said defined level. The stringent conditions are dependent on hybridization and washing conditions. These conditions may be modified according to methods known to those skilled in the art. Generally, high-stringency conditions are a hybridization temperature approximately 5° C. less than the melting point (Tm), preferably close to the Tm of the perfectly base-paired strands. The hybridization procedures are well-known in the art.

High-stringency conditions generally involve hybridization at a temperature of approximately 50° C. to approximately 68° C. in a 5×SSC/5×Denhardt's solution/1.0% SDS solution, and washing in a 0.2×SSC/0.1% SDS solution at a temperature between approximately 60° C. and approximately 68° C.

By “cells with high Kindlin-1 expression level”, it is meant cancer cells having a Kindlin-1 expression level in equal to, or above, a reference level.

As used herein, the term “reference level” means an expression level of Kindlin-1 which is determined for the methods or use of the invention. In one particular embodiment, the reference level is determined by the mean value of the Kindlin-1 expression level in several cancer cells samples. In another embodiment, the reference level is the optimal threshold value determined by ROC analysis. Cancer cells samples used for the determination of the reference level are preferably of the same type of cancer as the cancer cells of the subject. Preferably, the cancer cells used for determining the reference level originate from the same organ and same cellular type (preferably epithelial cells, i.e. carcinoma) as the cancer cells of the subject. According to the invention, a reference level may be determined by a plurality of samples, preferably more than 5, 50, 100, 200 or 500 samples.

For example, if the reference level is determined the mean value of the Kindlin-1 expression level measured in samples of cancer cells, and the measured Kindlin-1 expression level in cancer cells of the subject is above the reference level, then said cancer of the subject can be considered as comprising cells with high Kindlin-1 expression level.

In a particular embodiment, the cancer is an EGFR/RAS-driven cancer.

The term “EGFR/RAS pathway” refers to all the reactions and metabolites, which are produced by the activation of EGFR and/or the activation of RAS and the following sequence of reactions and metabolites. It includes in particular the RAS-RAF-MEK1/2-ERK1/2 signaling axis, i.e. RAS proteins (K-RAS, H-RAS, N-RAS), BRAF, MEK (MEK1/2), and MAPK (ERK1/2) proteins. The term “EGFR/RAS pathway” also includes the reactions required for the activation of EGFR and/or RAS.

The term “EGFR/RAS-driven cancer”, as used herein, corresponds to cancer wherein EGFR and/or RAS is highly expressed and/or cancer wherein EGFR and/or RAS are activated.

The present invention will be further illustrated by the figures and examples below.

### FIGURES

FIG. 1: Kindlin-1 binding to β1-integrin is required to mediate cell migration and invasion. A. Migration and invasion assays of 168FARN cells transfected as indicated, indexes are normalized as the fold change compared to ctrl-cells. B. Persistence and velocity tracking (***p<0.001). Error bars indicate means±SEM from three independent experiments.

FIG. 2: Kindlin-1 promotes metastasis in vivo. RT-PCR for the hygromycin resistance gene was performed to quantify the number of control, Kind1 or AAKind1 cells that reached secondary organs (Student t-test, *p<0.05).

FIG. 3: Kindpep1 reduces β1-integrin activation. Fluorescence activated sorter (FACS) histograms (top) and the corresponding bar graphs showing the mean fluorescence intensity (MFI) of cell-surface active β-1 integrin (9EG7 staining) of MDA MB 231 cells untreated, treated with Kindpep1 (200 μM) for 15 h or treated with 2 mM MnCl₂.

FIG. 4: Kindpep1 reduces cell survival, motility and invasion of breast cancer cells. MDA-MB-231 cells were treated with Kindpep1 (50, 100 or 200 μM). A. An MTS assay was performed and the absorbance was measured at 490 nm at different time points. B. A time-lapse imaging was performed for 16 h. Total distances were quantified and represented as the mean±SEM of values (n=30 cells tracked by condition). C. Plots shows overlays of the representative trajectories travelled by cells. D. Quantification of the matrix degradation area in control and Kindpep1 treated cells represented as the mean±SEM of values. E. A transwell cell invasion assay was performed for 6 h. Cells were then counterstained with DAPI and imaged with a fluorescence microscope. The number of invasive cells was quantified and represented as the mean±SEM of values.

Statistical analyses were performed by a t-test (****p<0.0001; **p<0.01; *p<0.05) Results are representative of experiments performed at least in duplicate.

FIG. 5: Kindpep1 reduces tumor growth and metastatic spread in breast cancer in vivo. A. Diagram presenting the course of the experiment. B. Corresponding relative tumor growth graph to bioluminescence images of tumors at day 25. C. Corresponding graph showing the quantification of the total area of metastatic nodules found in the different groups of mice to bioluminescence images of metastasis at day 25. Area ph/s/group stands for area photon/second/group.

FIG. 6: Kindpep1 reduces the metastatic spread in a breast cancer PDX model. A. Diagram presenting the course of the experiment. B Quantification of the number of metastatic nodules found in the lungs of control and Kindpep1 treated mice. Mean±SEM are represented. Statistical analyses were performed by a t-test.

FIG. 7: Kindlin-1 depletion specifically affects survival of KRAS-dependent cancer cells. KRAS or Kindlin-1 were silenced (4 and 7 days respectively) in H358 and H1975 lung cancer cell line. Then they were counterstained with DAPI and imaged with a fluorescence microscope for cell viability quantification. FIG. 7 represents bar graph of the average cell counted in these images.

FIG. 8: KRAS-dependent cell lines exhibit a higher sensitivity to Kindpep. A. Different breast, lung and pancreatic cancer cell lines (grey: RAS-dependent; black: RAS-independent cancer cell lines) were treated with Kindpep1. Then cell viability was assessed by CelltiterGlo assay. B. Dose-response curve and half maximal inhibitory concentration (IC50) values of Kindpep1 in different lung, pancreatic and bladder cancer cell lines (grey: RAS-dependent; black: RAS-independent cancer cell lines) treated with increasing doses of Kindpep1 for 72 h.

FIG. 9: Kindpep decreases tumor growth of KRAS-dependent tumors in vivo. A. Diagram presenting the course of the experiment. B. Scatter plot showing the tumor volume of the different mice of the control and Kindpep1 treated groups, at the end of the treatment. Mean±SD are represented (*p<0.05). C. Graph monitoring tumor growth of control and Kindpep1 treated mice during the course of the experiment. Mean±SD are represented.

FIG. 10: Domain, length and residues required for Kindpep1 cytotoxic activity. A dose response experiment was performed on H358 cell line for testing the cytotoxic activity of the different peptides derived from Kindpep1 sequence. A. The activity of the peptides were assessed by calculating the half-maximal inhibitory concentrations (IC50) for each peptide. B. Table summarizing the results obtained for the different tested peptides.

FIG. 11: Levels of phosphorylation of EGFR protein in BT-20 treated with or without Kindpep1 and with or without EGF. BT-20 cells were treated with or without Kindpep1 for 20 h. Cells were then starved overnight and treated with EGF (100 ng/ml for 15 min). Cellular extracts were immunoblotted with anti-pEGFR, and anti-actin antibodies.

FIG. 12: Immunostaining of EGFR in BT-20 treated with or without Kindpep1 and with or without EGF. BT-20 cells were treated with or without Kindpep1 for 20 h. Then, cells were starved overnight and treated or not with 2 ng/ml EGF for indicated times. Cells were immunostained with anti-EGFR (original magnification: ×60).

FIG. 13: Domain, length and residues required for Kindpep1 cytotoxic activity. BT-549 cells were transfected as indicated. Cell viability was assessed by CelltiterGlo assay and a dose-response curve and half maximal inhibitory concentration (IC50) values of Kindpep1 was assessed for 48 h.

### EXAMPLE

### Materials and Methods

### Identification and Synthesis of Peptides Targeting the Interaction Between Kindlin-1 and β-Integrin.

Overlapping dodecapeptides with two amino acid shifts, spanning the complete sequence of human Kindlin-1 protein, were prepared by automated spot synthesis on a nitrocellulose membrane. The membrane was saturated using 3% non-fat dry milk, 3% bovine serum albumin (BSA) for 2 h at room temperature and incubated with 3 μg/ml β1-integrin recombinant protein, for a hybridization overnight at 4° C. After several washing steps, the membrane was incubated with anti-31-integrin antibody for 2 h at room temperature, followed by horseradish peroxidase (HRP)-conjugated secondary antibody for 1 h at room temperature. Positive spots were visualized using the Enhanced chemiluminescence (ECL) system (Bio-Rad). The discovered Kindlin-1 sequences were combined with a Drug Phosphatase Technology (DPT) cell permeable shuttle in order to generate the Kindpeps interfering peptides.

All peptides were synthesized in a fully-automated synthesizer with a solid-phase procedure and standard Fmoc chemistry. The purity (>95%) and composition of the peptides were confirmed by reverse-phase high-performance liquid chromatography (HPLC) and mass spectrometry.

### Cell Lines

NCI-H358, NCI-H727, NCI-H1650, NCI-H1975 and Calu1 human lung cancer cell lines; AsPC-1, PANC-1, HPAF-II, MiaPaCa human pancreatic cancer cell lines; HT-1376, RT-4, UMUC3 human bladder cancer cell lines; MDA-MB-231, BT-20 and BT-549 human breast cancer cell line, and HEK293T cells were purchased from the American Type Culture Collection (ATCC). The bladder cancer cell line Ku1919 (DSMZ) and the human head and neck BIRC22 (ECACC), HsC4, OSC20, SAS and SAT cells (JCRB) cancer cell lines were purchased from different suppliers as indicated. Mouse tumor cell line 168 FARN and 4T1 were kindly provided by Dr. Fred Miller (Michigan Cancer Foundation, Detroit, MI). The cell lines are authenticated each 20 passages by using the GenePrint 10 System kit. Cells were grown in DMEM, RPMI 1640, MEM Alpha or Leibovitz's L-15 medium supplemented with 10% fetal bovine serum (FBS) and 1% antibiotics (50 μg/mL penicillin, 50 μg/mL streptomycin, 100 μg/mL neomycin) and maintained at 37° C. with 5% CO₂ (without CO₂ for Leibovitz's L-15 medium).

### Knockdown Experiments

Transfections were performed using JetPrime (Polyplus-transfection, New York, NY, USA) following the manufacturer's protocol with siRNA-negative control or siRNA-Kindlin- or siRNA-Kras from Dharmacon for transient silencing experiments.

### Expression Constructs and Transfections

The human KIND1 cDNA was subcloned into pIREShyg3 vectors as previously described. The Kindlin-1 PTB (PhosphoTyrosine-Binding domain) mutation QW611/612AA was introduced with a site-directed mutagenesis using the QuikChange site-directed mutagenesis kit, following the manufacturer's recommendations.

The primers used for mutagenesis were:

(SEQ ID NO: 18)
F5′-CATGGAGATTCACAAATATCAAAGCGGCGAATGTAAACTGGGAAA
CCCGGC-3′,
and
(SEQ ID NO: 19)
R3′-GTACCTCTAAGTGTTTATAGTTTCGCCGCTTACATTTGACCCTTT
GGGCCG-5′.

All cDNA constructs were verified by sequencing. Transfections were performed using Lipofectamine Reagent following the manufacturer's instructions. Stable transfectants were grown in the presence of 200 μg/mL hygromycin.

### Western Blotting

Cells were lysed using RIPA buffer (50 mM Tris-HCl, pH 8; 150 mM NaCl; 0.5% triton; 0.5% deoxycholic acid) containing protease inhibitors (1:1000 orthovanadate, 1:1000 apoprotinine, 1:200 PMSF). Protein extracts were loaded on a polyacrylamide gel, transferred to a nitrocellulose membrane and incubated overnight at 4° C. with the following primary antibodies: rabbit anti-Kindlin-1 (1:10000); 31-integrin (mouse anti-(1-integrin 18/CD29 clone, 1:2500); pEGFR-Y1068 (1:1000, D7A5); pEGFR-Y1173 (1:1000, 53A5); β-actin (20000, A3854) or GAPDH (1:1000) used as loading control. The signals were detected according to the ECL Western Blotting Analysis System procedure.

### Co-Immunoprecipitation

Cells were lysed using NP40 buffer (50 mM Tris-HCl, pH 7.5; 150 mM NaCl; 0.5% NP40) containing protease inhibitors (1:1000 orthovanadate, 1:1000 apoprotinine, 1:200 PMSF). Protein extracts were incubated with 1 μg antibodies for Kindlin-1, β1-integrin or normal rabbit IgG and 10 μl Sepharose Protein A beads with rotation at 4° C. overnight. Beads were washed with NP40 buffer three times and immunoprecipitates were resolved by western blotting.

### Flow Cytometry Analysis

To quantify the expression levels of integrins, cultured cells were trypsinized, washed and incubated for 1 h at 4° C. in phosphate-buffered saline (PBS) containing 150 mM NaCl and 1% BSA with the rat anti-1 integrin (9EG7 clone, 1:300) primary antibodies obtained from BD Biosciences. Cells were then washed 3 times, incubated with Alexa Fluor 488-conjugated secondary antibody for 1 h at 4° C. and washed. 1×10⁴ cells were analyzed on a FACS Calibur. The mean fluorescence intensity of each sample was obtained using the Flowing software 2.

### 2D Migration/Invasion Assay

Transwell migration assays were performed in triplicate using cell culture inserts with 8.0-μm pore size membranes according to the manufacturer's protocol. 3×10⁴ cells were plated in the top chamber in Dulbecco's Minimal Essential Medium. In the bottom chamber, culture medium with 10% FBS was used as a chemoattractant. 24 h later, unmigrated cells were removed from the top of the membranes using cotton swabs and migrated cells were fixed and stained using crystal violet. To quantify the number of migrated cells, total membrane area was taken at 4× magnification using a light microscope. The number of cells per image was counted using ImageJ software. For transwell invasion assays, the top chamber was precoated with 4 μg/cm² of Matrigel.

### 3D Collagen Invasion Assay

2×10³ cells were allowed to form multicellular spheroids using the hanging droplet method. After 3 days, spheroids were embedded in type I collagen extracted from rat tail tendon (2.2 mg/ml final concentration). Spheroids were fixed in 4% paraformaldehyde immediately after matrix polymerization or after 4 days of invasion. After fixation, cells were permeabilized for 15 min in 0.1% Triton X-100/PBS and labelled with Alexa Fluor-545-phalloidin and DAPI. Labelled spheroids were imaged using inverted confocal laser scanning microscope LSM510 with 25× multi-immersion water/oil objective and collected as stack of images along the z-axis with a 10 μm interval between optical sections.

### Live-Cell Imaging

Migration assays were conducted on an Eclipse Ti-E inverted full-motorized microscope equipped with an incubation chamber maintained at 37° C. with 5% CO₂. Movies were acquired by an ORCA Flash 4.0 V2 digital CMOS camera controlled by NIS-Elements BR 3.0 software. Cell migration was recorded for 24 hours. Single cells' tracking was conducted using the “Manual Tracking” plugin of ImageJ software.

For 3 dimensions (3D) invasion assays, 24 hours after embedding in collagen matrix, spheroids were imaged every 15 min for 24 h with the CFI Plan Fluor DL 10× objective (NA 0.3) of an Eclipse Ti Inverted Microscope equipped with a CCD Coolsnap HQ2 camera. Images were collected as a stack of 15 μm interval. Focussed images were selected using Metamorph and migratory parameters were computed with ImageJ. Random motility was quantified by determining the ratio of the shortest, linear distance from the starting point of a time-lapse recording to the end point (D) compared with the total distance traversed by the cell (T).

### Immunofluorescence

Cells were seeded in 24-well plates pre-coated with 2 μg/cm2 fibronectin, incubated with PBS containing 1.5% BSA, and fixed for 10 min with PBS containing 4% paraformaldehyde. Cells were incubated with the anti-β1 integrin 9EG7 (1:300) for 45 min at room temperature under vibrations (450 rpm). Cells were then washed and incubated with alexa fluor-conjugated secondary antibodies, at room temperature for 30 min. F-actin was labeled using phalloidin conjugated to TRITC at room temperature for 30 min. Cells were then stained with PBS containing 0.2 μg/ml DAPI to visualize the nuclei. Sections were washed in 0.07 M PBS, mounted with Progold antifading and examined with a fluorescence microscope.

### Gelatin-FITC

Cells treated with or without peptides (100 μM) were cultured on FITC-gelatin-coated coverslips for 24 h. Cells were fixed and permeabilized in 4% paraformaldehyde, 0.3% Triton X100 during 10 min. Cells were then rinsed in PBS incubated with an anti-cortactin antibody-Alexa Fluor 647 Conjugated. To visualize F-actin, the cells were stained with Alexa-568-conjugated phalloidin.

### KI67 Immunohistochemistry

Murine tumor paraffin sections were prepared as previously described. Briefly, tumor blocks were deparaffinized, treated with 3% H₂O₂, and incubated with KI67 antibody (8D5 clone, 1/500, Cell signaling). The staining signals were revealed with Dako Real Detection Peroxidase/AEC kit. The slides were counterstained with Mayer's hematoxylin.

### Cell Viability Assay

7×10³ cells were seeded in 96-well plates with transparent bottom and incubated at 37° C., 5% CO₂ for 16 hours. Serially-diluted Kindpep1, Kindpep2 or PBS was added to the cells, and plates were incubated at 37° C., 5% CO₂ for 72 hours. Cell viability was assessed by CellTiter-Glo® Luminescent Cell Viability Assay kit according to manufacturer's protocol. The luminescence signal of treated wells were normalized to the appropriate PBS control wells in order to assess viability

### Mouse Studies

All procedures involving animals, including housing and care, method of euthanasia, and experimental protocols were conducted according to French veterinary guidelines and in accordance with a code of practice established by the local ethical committee (Curie institute or Université Claude Bernard (Lyon) guidelines on animal care).

168FARN model: 1×10⁶ cells (control, Kind1 and AAKind1) were injected subcutaneously into one mammary gland of 10-week old female BALB/c mice (n=10 for control, Kind1 and AAKind1). Mice were sacrificed when primary tumors reached the size of 20 mm³ or when moribund. Mammary glands, tumors and metastases were removed, measured using caliper and fixed using 10% buffered formalin. Paraffin-embedded tissues were prepared through standard techniques. Tumor volumes were calculated as: Volume (cm³)=a×b²/2 (a and b are the two registered perpendicular diameters a>b). Organs sections were stained with Mayer's hematoxylin.

4T1-luc model: 1×10⁵ 4T1-luc cells were inoculated subcutaneously in the flanks of 6-week old female BALB/c mice (n=28). Eleven days after tumor cell inoculation, immunodeficient mice were randomly assigned to receive intraperitoneal injection of Kindpep1 (2, 10 or 50 mg/kg) or placebo (PBS). The tumor growth and the progression of metastatic lesions were also weekly monitored by bioluminescence imaging of animals using the Nightowl imaging system. On day 25 after tumor cell inoculation, anesthetized mice were sacrificed by cervical dislocation.

NCI-H358 model: 1×10⁵ cells were injected subcutaneously in the flanks of 6-week old female BALB/c mice (n=10 mice for each control and treated groups). Tumor volume was measured with caliper 3 times a week. Treatment with Kindpep1 (10 mg/kg) or placebo (PBS) started when primary tumors reached the size of 150 mm³. The intraperitoneal treatment was performed 5 times per week until sacrifice (at J55). Tumor volumes were calculated as: Volume (cm³)=a×b²/2 (a and b are the two registered perpendicular diameters a>b). Tumor growth inhibition (TGI) was calculated using the following formula [1−(V_{ft}−V₀ₜ)/(V_{fc}−V_{0c}))]*100 where V_{ft}=final volume of the treated group (at the end of the treatment); Vot=initial volume of the treatment group (at the beginning of the treatment); V_{fc}=final volume of the control group (at the end of the treatment); V_{0c}=initial volume of the control group (at the beginning of the treatment).

### Breast Cancer Patient Derived Xenografts (PDX)

The triple negative breast cancer PDX model expressing high levels of Kindlin-1 was obtained from the Laboratoire d'Investigation Pré-clinique. Informed consent was obtained from patient before xenograft establishment. When tumor reached a volume of 60 to 200 mm³, mice were randomly assigned to the control or treated group. Kindpep1 was administrated by intraperitoneal injection at a dose of 10 mg/kg 5 times a week. The vehicle alone (Nacl 0.9%) was administered to the control group following the same treatment regimen as with the drug. The control and treated group consisted of 10 mice each.

### Results

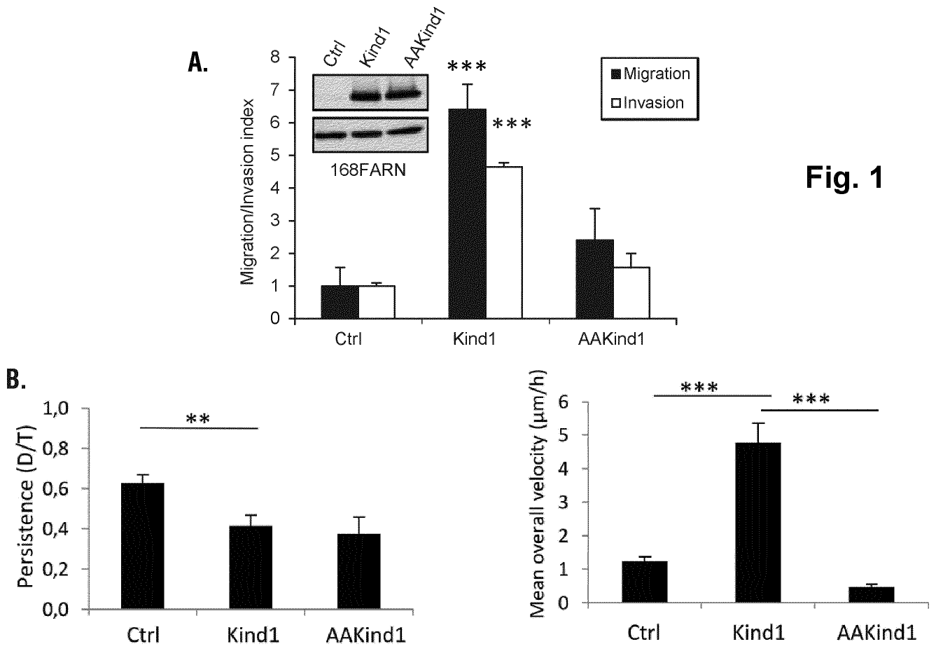

Kindlin-1 binding to β1-integrin is required to mediate cell migration and invasion Inventors had previously demonstrated that depletion of Kindlin-1 in 4T1 cells decreased cell migration and invasion. To test whether these phenotypic changes are dependent on integrin signaling, the migratory and invasive capacities of breast cancer cells expressing wild-type Kindlin-1 or an integrin-binding-defective mutant (hereafter cited as AAKind1) was analyzed. Ectopic expression of Kindlin-1 increased both migration and invasion in two-dimensional matrix experiments (FIG. 1A). Moreover, culturing the 168FARN tumor cells in a three-dimensional (3D) environment further highlighted the invasive properties induced by the expression of the different forms of Kindlin-1. Whereas control cells migrated relatively straight ahead (directional migration), cells expressing wildtype Kindlin-1 showed a different migration pattern characterized by the capacity of randomly changing directions. Time-lapse imaging and tracking of these cells allowed quantifying the effect of Kindlin-1 expression on cell motility. The persistence (the ratio of displacement and total distance travelled) of individual cells during random migration on collagen matrix was measured. This D/T directionality ratio was reduced in cells expressing wildtype Kindlin-1 (FIG. 1B; p=0.007, Student's t-test), accompanied by a 3.8-fold increase in the velocity of cell migration (p=6.10⁻⁵, Student's t-test). Thus, cells expressing Kindlin-1 while travelling a similar distance in 3D environment had an increased ability to persistently migrate in multiple directions compared to control cells (FIG. 1B). Inventors also investigated whether the Kindlin-1-mediated cell migration depends on a direct interaction of Kindlin-1 with the integrin β-tail. Ectopic expression of AAKind1, did not trigger migration through 2D and 3D matrices (FIG. 1A). These data indicate that Kindlin-1-mediated migration requires an intact binding domain, suggesting that interaction with integrins is necessary for Kindlin-1 to promote migration. Strikingly, the mutant form of Kindlin-1 completely abolished the migration capacities of the transfected 168FARN cells in 3D environment (as compared to control cells). These results demonstrate that the replacement of two amino acids in Kindlin-1 generated a mutant protein that acts as a dominant negative in preventing cell migration and invasion.

### Kindlin-1-Dependent Integrin Activation is Required for Local Invasion and Metastasis

To further investigate whether Kindlin-1 is sufficient to trigger invasion in vivo, orthotopic injection of wild-type Kind1 or AAKind1 cells into the mammary fat pad of syngeneic BALB/c mice was performed. While 25% of the mice injected with the control cells remained free of disease (55 post-injection days), all mice injected with 168FARN cells expressing either wild-type or mutant forms of Kindlin-1 formed primary mammary tumors. The aggressiveness of the Kind1 or AAKind1 cells was compared by analyzing the disease-free survival rate of injected mice. Mice injected with both cells showed shorter lag time before disease occurrence and had significantly reduced survival as compared with the parental cell line.

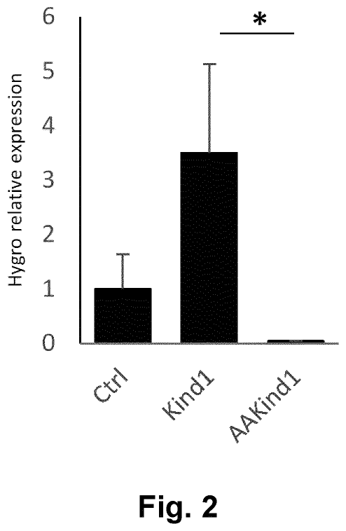

After sacrifice, tumor masses were excised from mice bearing either ctrl, Kind1 and AAKind1 tumors. Subsequent sectioning, staining and pathologic examination of these sections revealed distinct invasive capacities in Kind1 and AAKind1 cells. Kind1-expressing tumors exhibited increased local invasion of the surrounding normal mammary tissue, whereas AAKind1 tumors grew focally as encapsulated masses. AAKind tumors exhibited no invasion into the neighboring healthy muscles and instead maintained a distinct margin with the normal tissue. Of note, examination of the primary tumors demonstrated that Kind1 and AAKind1 tumors exhibited a similar proliferation rate as quantified by Ki67 staining in tumor sections. To further investigate a causal role for Kind1 in breast cancer progression, the effect of Kind1 or AAKind1 expression on metastasis was examined. After sacrifice, distant organs were collected from the three groups of mice. Kindlin-1 expression increased the metastatic spread of tumors to the different secondary sites. Furthermore, the qRT-PCR amplification of the hygromycin-resistance gene, revealing the expression of transfection vector to quantify the number of cells that reached secondary organs, showed an increased metastatic spread in Kind1 tumors, whereas AAKind1 tumors exhibited no metastases in secondary organs (FIG. 2). Thus, in accordance with in vitro results mutant cells did not have the capacity to migrate and metastasize in vivo.

All together, these results demonstrated that expression of Kind1 could promote tumor metastasis in vivo, and this capacity was abrogated by the mutation of the integrin-binding domain of the protein.

### Development of a Cell Penetrating Peptide Targeting Kindlin1-Integrin Interaction

To determine the residues of Kindlin-1 that interacted with β1-integrin, a nitrocellulose membrane with peptides corresponding to the sequence of human Kindlin-1 was generated. Kindlin-1 membrane was then incubated with β1-integrin recombinant proteins. Hybridization using specific antibodies allowed the identification of two sets of spots corresponding to two different sequences of Kindlin-1. These sequences were then combined to an optimized cell penetrating sequence previously developed, Mut3DPT (VKKKKIKAEIKI, SEQ ID NO: 17), to obtain two different cell penetrating peptides: Kindpep1 (corresponding to the sequence SEQ ID NO: 15) and Kindpep2 (corresponding to the amino acid sequence SEQ ID NO: 16).

As the three Kindlins share a high sequence homology, the sequence of Kindpep1 and Kindpep2 was compared with Kindlin-2 and Kindlin-3 sequences. Significant similarities were not found with Kindpep1 and a low homology with Kindpep2, suggesting that Kindpep sequences were exclusive from Kindlin-1.

Then, the inventors were interested in confirming that the specific target of Kindpep1 was the Kindlin-1/β1-integrin complex. To this end, it was analyzed whether the peptide was able to target and disrupt the interaction of Kindlin-1 and 31-integrin in vitro. HEK293 cells were transiently transfected with Flag-Kindlin-1 and Myc-β1-integrin, alone or in combination. For the competition assay, cell lysates from control untreated or Kindpep1-treated cells (100 μM, 24 h) expressing Flag-Kindlin-1 and Myc-β1-integrin were immunoprecipitated with anti-Kindlin-1 antibody and the presence of the Kindlin-1/β1-integrin complex was analyzed by western blot. 31-integrin was detected in control Kindlin-1 immunoprecipitate, while it was undetectable after competition with 100 UM of Kindpep1. This result validates that Kindpep1 targets and is able to disrupt the interaction of Kindlin-1 and β1-integrin in vitro.

### Kindpeps Inhibited the Capacity of Kindlin-1 to Activate Integrins in Breast Cancer Cells

Since Kindlin-1 is known to be a major regulator of β-integrin activation, the inventors then wanted to analyze whether the peptides could reduce Kindlin-1-mediated B1-integrin activation. Immunofluorescence labeling of 168FARN-Kind1 cells was performed using the 9EG7 antibody, which specifically recognizes the activated form of β1-integrins. Active B1-integrin localized at the multiple focal adhesion sites exhibited by the 168FARN cells expressing Kindlin-1. These cells were incubated with Kindpeps 400 UM, for 48 hours and the immunofluorescence images show a drastic reduction of active β1-integrin accompanied by a morphological change, cells are less spread and adopt a more filamentous shape.

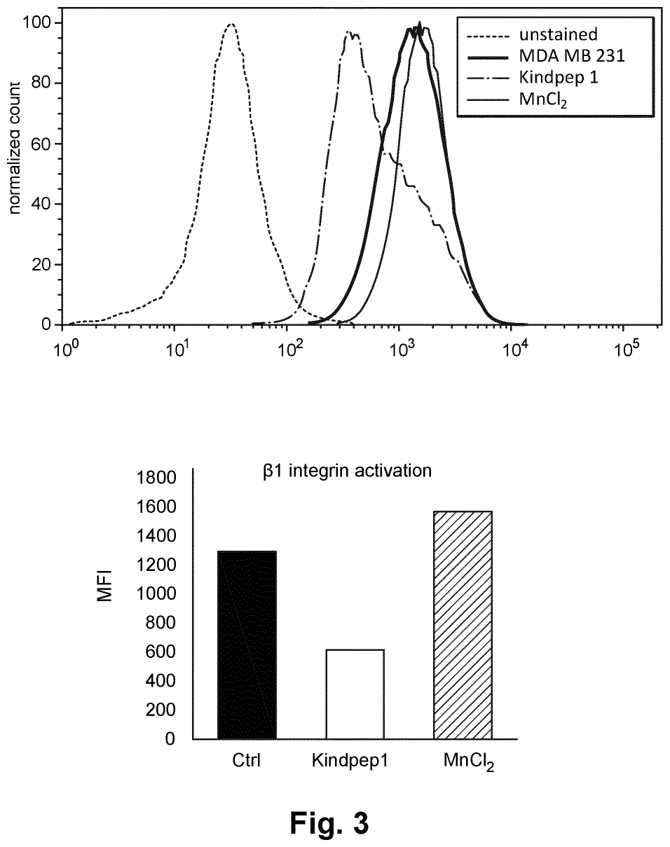

The effect of Kindpep1 on β1-integrin activation was confirmed by performing the same immunofluorescence assays using the human breast cancer cell line MDA MB 231, characterized by a high Kindlin-1 protein endogeneous expression. Of note, even with half Kindpep1 concentration (200 μM) and shortened the treatment time to 15 hours in these cells, it was still possible to observe a flagrant effect on the reduction of β1-integrin activation. To confirm these results, inventors performed a flow cytometry assay using the 9EG7 in MDA MB 231 cells incubated with Kindpep1 200 μM. As expected, Kindpep1 significantly reduced the levels of active β1-integrin on the cell surface (FIG. 3). MnCl₂, an activator of β-integrins, was used as a positive control and MDA MB 231 cells treated with MnCl₂ presented increase levels of active β1-integrin, as shown in FIG. 3.

Altogether, these results demonstrate the ability of Kindpeps to disrupt Kindlin-1/β1-integrin complex, drastically reducing the activation of β1-integrin. Thus, confirming the importance of Kindlin-1 on integrin activation.

### Kindpeps Impaired Cell Survival, Motility and Invasion in Breast Cancer Cells

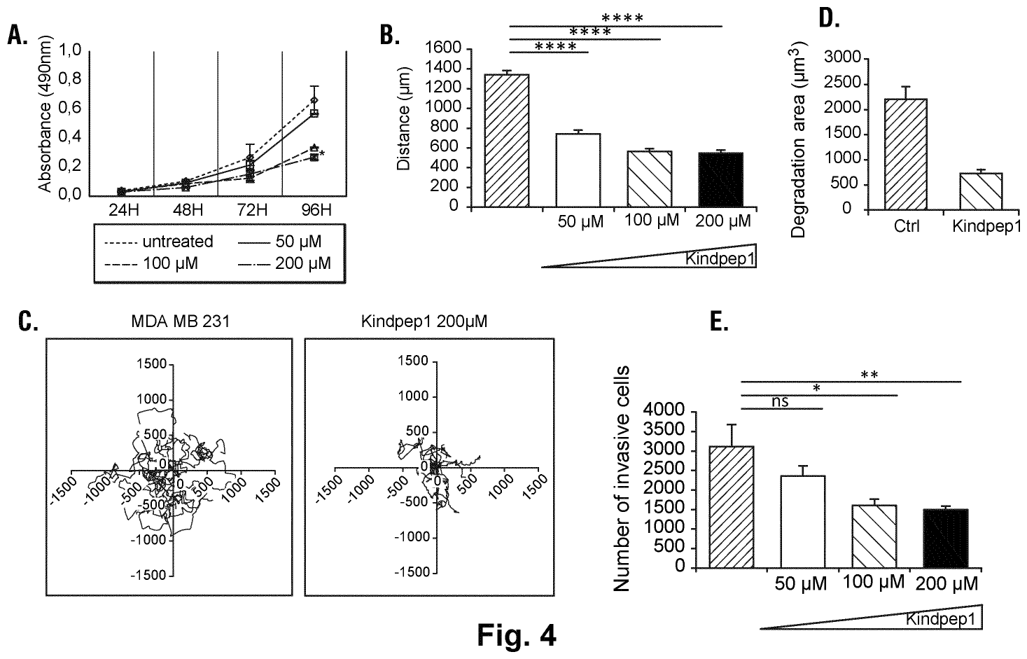

The ability of Kindpep1 to reduce the activation of β1-integrin translated into an impact on integrin-mediated pro-tumorigenic activities in vitro was investigated. First, the effect of Kindpep1 on the proliferation of breast cancer cells was analyzed. MDA MB 231 were treated with increasing concentrations of Kindpep1 (50, 100 and 200 μM) and proliferation was assessed by an MTS assay every 24 hours, for 96 hours. As shown in FIG. 4A, Kindpep1 significantly impacted cell proliferation at 100 and 200 UM but the effect was minimal at 50 μM. Next, the impact on cell migration was explored. MDA MB 231 cells treated with increasing concentrations of Kindpep1 (50, 100 and 200 μM) were monitored during 16 hours in a time-lapse microscopy experiment. FIGS. 4B and C show that cell migration was significantly impaired, already with the smallest concentration of Kindpep1.

The capacity of these cells to degrade the extracellular matrix and perform invasion was analyzed. First, the ability of cells to degrade the matrix was measured by culturing them on a cross-linked fluorophore (FITC)-conjugated-gelatin matrix-coated coverslips for 24 hours with or without Kindpep1 at 100 μM. Cells were then stained with phalloidin for actin. Inventors could notice that Kindpep1 considerably reduced the amount of matrix degradation as compared with control cells. The dark (non-fluorescence) areas of degradation observed in control cells were much more reduced when cells were treated with Kindpep1. Indeed, the mean degradation area of control cells was 2204 μm³ versus 729 μm³ in the case of Kindpep1 treated cells (FIG. 4D). Finally, a transwell invasion assay was performed to analyze the invasive capacities of cells treated with increasing concentrations of Kindpep1, as already done for migration. A significant decrease in the number of invasive cells was observed at 100 and 200 μM (FIG. 4E).

Altogether, these findings demonstrate that the ability of Kindpep1 to disrupt Kindlin-1/1-integrin complex, drastically reducing the activation of 31-integrin, leads to a substantial reduction in the capabilities of breast cancer cells to proliferate, migrate, degrade the extracellular matrix and invade, in vitro.

### In Vivo Effects of Kindpep1 on Breast Cancer Cells

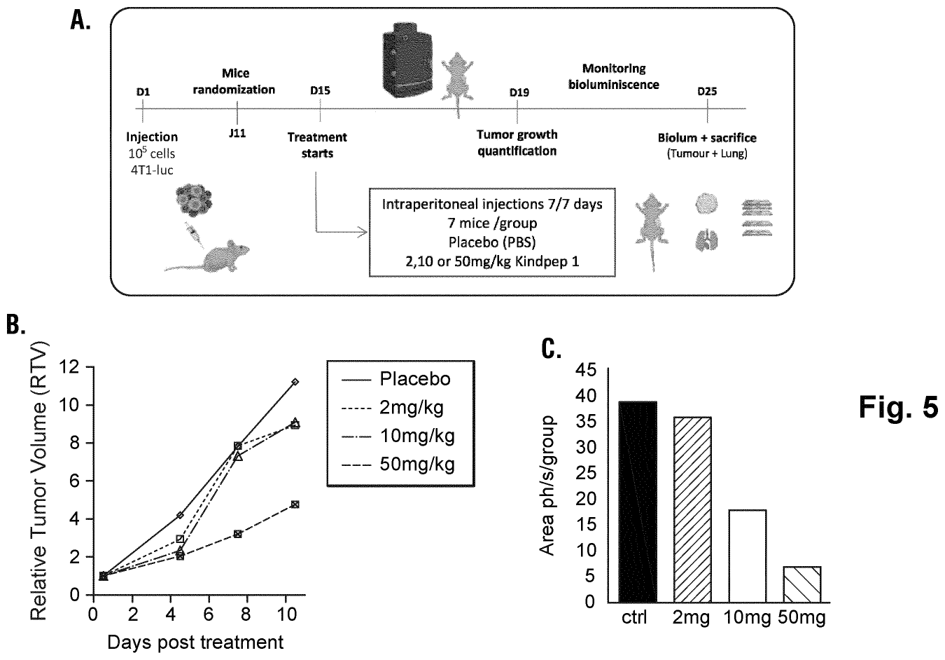

These observations prompted the investigation of the effects of Kindpep1 in vivo. As inventors had previously demonstrated the major role of Kindlin-1 in the development of lung metastasis in breast cancer, they focused on the metastatic process. Thus, the effects of Kindpep1 on tumor dissemination in a metastatic breast cancer model was studied. 4T1 cells were used because they have a high Kindlin-1 expression and have been also described to form lung metastasis in vivo. Moreover, luciferase-expressing cells were used to take advantage of the bioluminescent properties and track these cells over time in live animals. As shown in FIG. 5A, 10⁵ 4T1 cells were subcutaneously injected in 28 BALB/c mice, 10 days after mice were divided into 4 groups of treatment receiving placebo (PBS), 2, 10 or 50 mg/kg Kindpep1 7 day per week during 10 days. During the course of the experiment tumor growth and the development of metastasis were controlled by non-invasive bioluminescence imaging. Importantly, no death was observed during the treatment and no any side effects was detected of Kindpep1 in any of the treated mice. At the end of the experiment, relative tumor volume was calculated and a decrease in tumor growth when mice were treated with 50 mg/kg of Kindpep1 was observed (FIG. 5B). Relatively to the development of metastasis, a dose-dependent effect of Kindpep1 was observed (FIG. 5C).

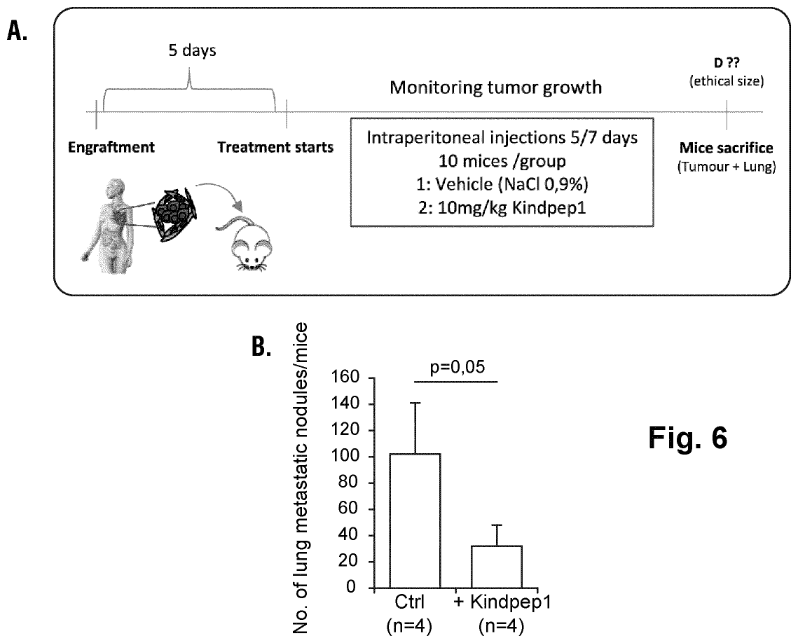

Then, a triple negative breast cancer patient derived xenograft (PDX) model previously characterized as highly metastatic and presenting high expression levels of Kindlin-1 was chosen. Tumor fragments were grafted into the interscapular fat pad of female nude immunodeficient mice and 5 days later the treatment started. Mice were treated with 10 mg/Kg of Kindpep1 or the vehicle (NaCl 0.9%) alone as negative control. The treatment was administered by intraperitoneal injections 5 days per week and tumor growth was monitored until reaching the ethical size, i.e. 2 cm³ (FIG. 6A). Importantly, no death was observed during the treatment and no side effects were detected in all treated mice.

Once tumors reached the ethical size, mice were sacrificed and lungs were collected to perform immunohistochemical analysis and evaluate the presence of metastasis. A reduction in the number of metastatic nodules in the group of mice treated with Kindpep1 as compared to the controls was observed (FIG. 6B).

### Specificity of Kindpep1 in EGFR and Ras-Driven Cancers

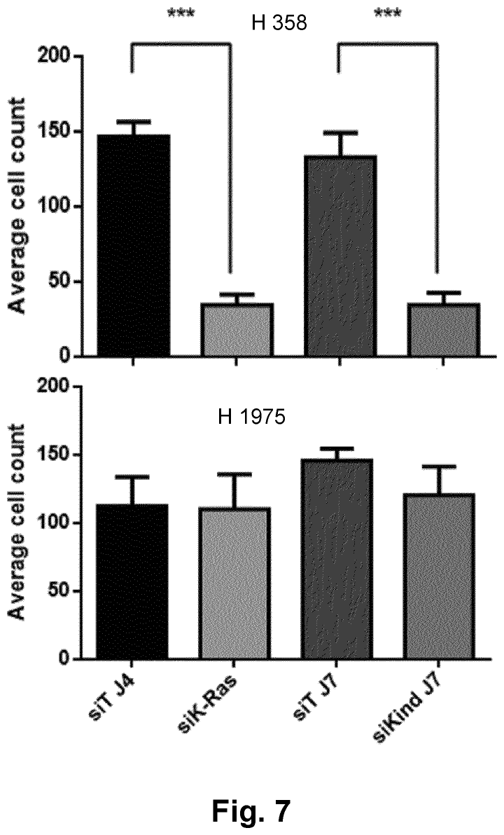

Inventors have previously demonstrated that the RAS/MAPK pathway is enriched in cancer cell lines with a high expression of Kindlin-1. In addition, GSEA plots highlighted the KRAS dependency gene set to be enriched in cancer cell lines with high levels of Kindlin-1 (see international application WO2021/228834 A1). Thus, Kindlin-1 was depleted in the pancreatic and lung cancer KRAS-dependent cell lines ASPC1 and H358 and in the KRAS-independent cell lines, PANC1 and Calu-1 to check the effect on cell survival. The depletion of Kindlin-1 drastically affected the survival of KRAS-dependent cells whereas it did not have any impact in the viability of KRAS-independent cells. Moreover, silencing Kindlin-1 had the same effect as silencing KRAS in the KRAS-dependent lung cancer cell line H358 and no effect on the H1975 independent cell line as shown in FIG. 7. These results suggest that KRAS-dependent cells need Kindlin-1 to survive.

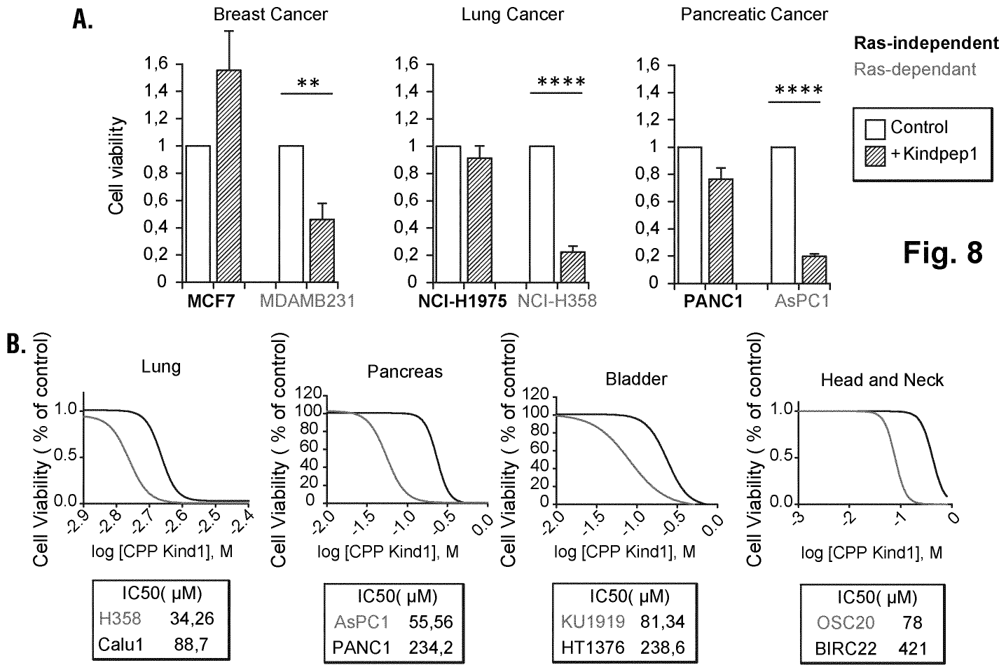

In order to evaluate the effect of Kindpep1 in EGFR and KRAS-dependent versus independent cell lines, one dependent and one independent cancer cell line were chosen representing lung, pancreatic, bladder, and head and neck cancers known to be either EGFR or RAS-driven carcinomas as described before (see international application WO2021/228834 A1). The different cell lines were treated with increasing concentrations of Kindpep1. As shown in FIG. 8A, only dependent cells were sensitive to Kindpep1, presenting a strong reduction of cell viability. Further experiments were performed for additional KRAS-dependent vs-independent (HPAFII vs MiaPaCa pancreatic cell lines; H727 vs. H1650 lung cancer cell lines; RT4 vs. UMUC3 bladder cancer cell lines; and Colo 320DM vs Colo205 colon cancer cell lines) and similar results were obtained (data not shown).

Inventors then sought to establish the Kindpep1 IC50 for each one of the cell lines. A range of Kindpep1 concentrations was used to treat the cells. After 72 hours a viability assay was performed. As expected, EGFR and KRAS-dependent cell lines presented significantly lower IC50, meaning a stronger sensitivity to Kindpep1 (FIG. 8B). Taken together, these findings suggest Kindlin-1 is an oncogene vulnerability of EGFR and KRAS-dependent cancer cell lines and indicate that Kindpep1 specifically affects the viability of these cells.

### In Vivo Effects of Kindpep1 on a KRAS-Dependent Lung Cancer Xenograft Mouse Model

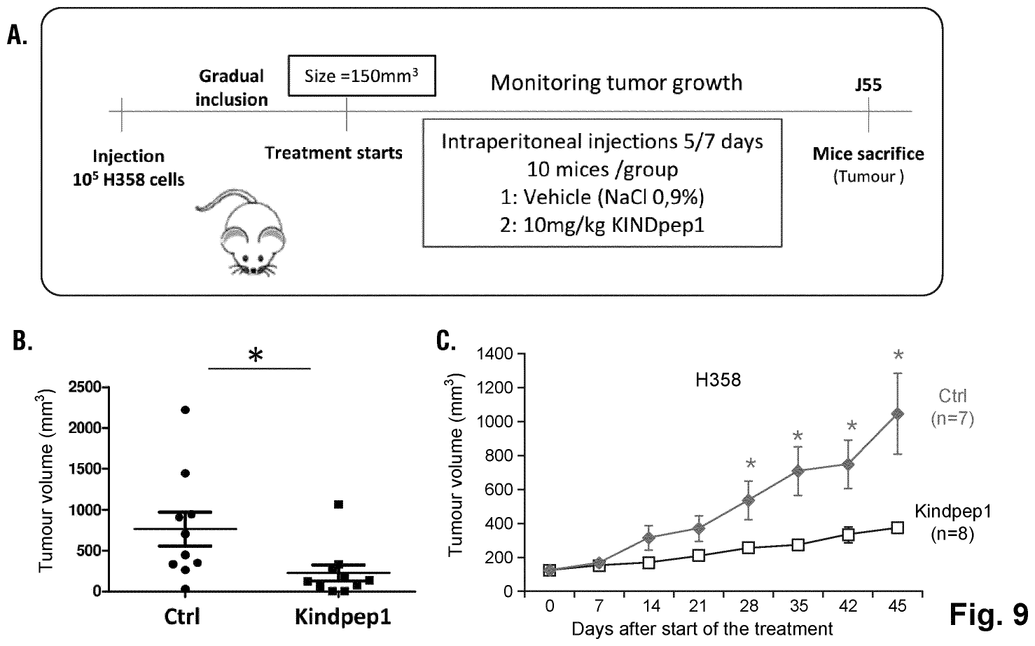

Finally, inventors wanted to validate in vivo the impact of Kindpep1 treatment in KRAS-dependent tumors. The H358 lung cancer cell line was used as model of study. 10⁵ cells were engrafted in nude mice and treatment started when tumors reached a size of 150 mm³. At that point, mice were treated with 10 mg/Kg of Kindpep1 or the vehicle (NaCl 0.9%) alone as negative control. The treatment was administered by intraperitoneal injections 5 days per week and tumor growth was monitored for 50 days (FIG. 9A). At the end of the treatment, Kindpep1 induced a significant decrease in tumor growth as the mean size of tumors was 1042 mm³ in the control group compared to 372 mm³ in the treated group (p=0.01; FIG. 9B-C). Kindpep1 had a high in vivo antitumor efficacy as measured by a TGI of 72.7% without apparent toxicities in nude mice. Thus, these results confirm that Kindpep1 is able to induce in vivo tumor growth inhibition in a KRAS-dependent lung cancer model.

### Domains or Residues Required for Biological Activity of Kindpep1

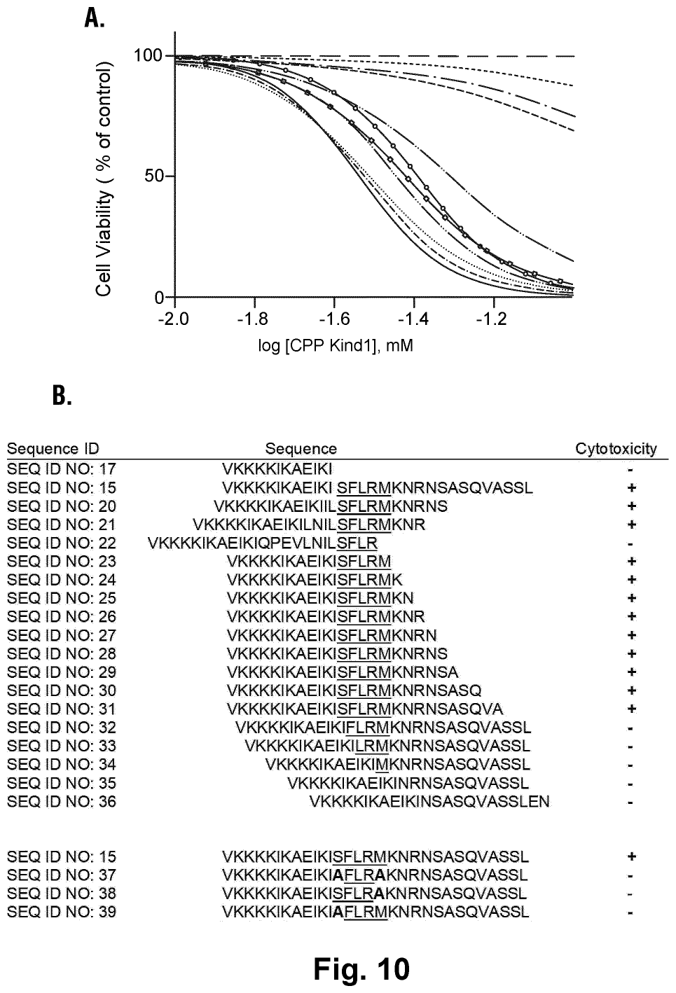

The minimal sequence required for the biological activities of Kindpep1 was studied by shortening kindpep1 sequence both from N and C terminus. The cytotoxic activity of all peptide derivatives were tested on the H358 lung cancer cell line in a dose dependent manner. The IC50 of the active peptides was determined (FIG. 10A) and the domain retaining the biological activity was the sequence: SFLRM highlighted in FIG. 10B. In addition, inventors generated several mutant peptides in which the AA of the domain of activity were replaced by alanine residues (S₄₉₁ and M₄₉₅) and tested their ability to impair H358 cell viability. Residue S₄₉₁ and M₄₉₅ of Kindlin-1 were required for Kindpep1 cytotoxic capabilities.

### Kindpep1 Inhibits EGFR Signaling in Breast Cancer Cells

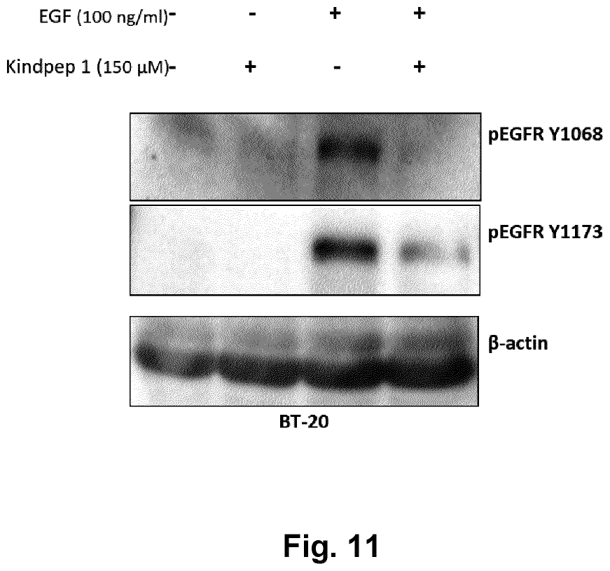

Upon EGF stimulation, EGFR receptor is phosphorylated at positions Y1068 and Y1173, which induces the activation of EGFR pathway. Interestingly, treatment of BT-20 cells with Kindpep 1 resulted in an impaired response to EGF as shown by the decreased levels of phosphorylation of EGFR protein (FIG. 11).

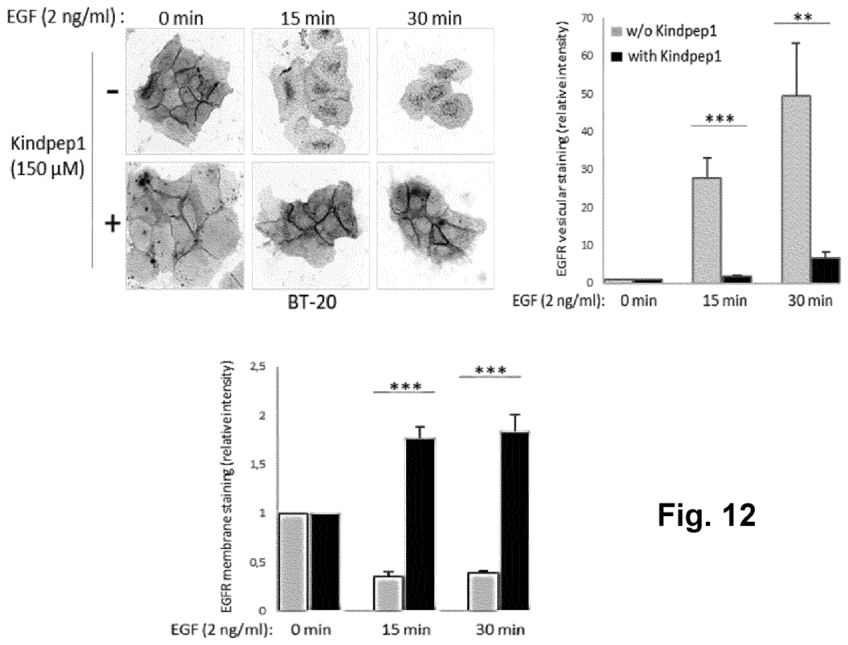

Furthermore, Kindpep1 impaired the internalization of the receptor. Upon Kindpep1 treatment, EGFR remained at the plasma membrane and is no longer localized in intracellular vesicles as compared to control cells, suggesting an effect of Kindpep1 on EGFR trafficking (FIG. 12).

### The Cytotoxicity of Kindpep1 Depends on Kindlin-1 Protein Expression.

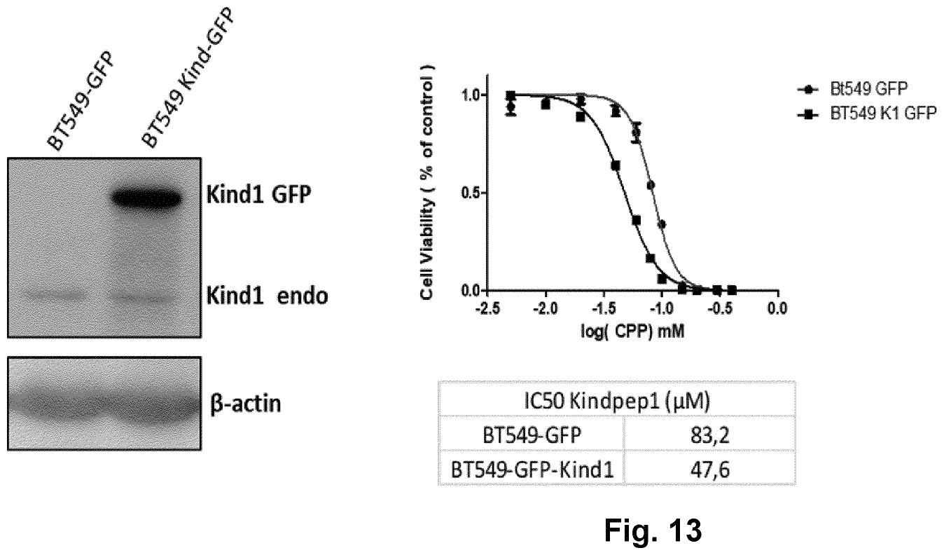

BT-549 breast cancer cells were transfected with an expression vector allowing ectopic expression of a GFP-tagged kindlin-1. The results (FIG. 13) showed that the ectopic expression of Kindlin-1 enhance the sensitivity of BT-549 cells to the cytotoxic effect of Kindpep1. The half maximal inhibitory concentration (IC50) values of Kindpep1 were reduced (two-fold) in cells overexpressing Kindlin-1 as compared to control cells.

### CONCLUSIONS

Inventors generated new peptide mimics of Kindlin-1, Kindpep1 and Kindpep2, which specifically binds β1-integrin disrupting its interaction with Kindlin-1. The disruption of this interaction drastically reduces the activation of β1-integrin, leading to a strong reduction in the capacities of breast cancer cells to proliferate, migrate, degrade the extracellular matrix and invade, in vitro; and to generate lung metastasis in vivo. In addition, Kindpeps specifically induces cell death in EGFR and KRAS-dependent cancer cell lines of diverse epithelial tissue origins.

Altogether, these results strongly suggest that interfering with Kindlin-1/β1-integrin interaction could be a promising and innovative therapeutic approach for the selective targeting of EGFR and RAS-driven epithelial cancers.

1-25. (canceled)

26. An isolated peptide comprising the amino acid sequence SEQ ID NO: 1:
(SEQ ID NO: 1)
SFLRM
wherein said isolated peptide comprises from 5 to 50 amino acids, and said isolated peptide does not consist of sequence PRRSFLRMP (SEQ ID NO:40).

27. The peptide according to claim 26, wherein
i) it further comprises at least one amino acid at the carboxyl-terminal end of sequence SEQ ID NO: 1 and/or
ii) the first amino acid directly after the carboxyl-terminal end of sequence SEQ ID NO: 1 is a lysine and/or iii) it comprises at least 7 consecutive amino acid sequence of SEQ ID NO:2: LNILSFLRMKNRNSASQVASSL (SEQ ID NO: 2), and/or
iv) said peptide has at least 75% of identity with the amino acid sequence SEQ ID NO: 2.

28. The peptide according to claim 26, wherein said peptide is selected from the group consisting of the peptides of amino acid sequences:
```
(SEQ ID NO: 3)
SFLRMKNRNSASQVASSL,
  (SEQ ID NO: 4)
SFLRMKNRNSASQVA,
  (SEQ ID NO: 5)
SFLRMKNRNSASQ,
  (SEQ ID NO: 6)
ILSFLRMKNRNS,
  (SEQ ID NO: 7)
LNILSFLRMKNR,
  (SEQ ID NO: 8)
SFLRMKNRNSA,
  (SEQ ID NO: 9)
SFLRMKNRNS,
  (SEQ ID NO: 10)
SFLRMKNRN,
  (SEQ ID NO: 11)
SFLRMKNR,
  (SEQ ID NO: 12)
SFLRMKN,
  (SEQ ID NO: 13)
SFLRMK,
and
  (SEQ ID NO: 1)
SFLRM.
```

29. An isolated peptide comprising the amino acid sequence SEQ ID NO: 14:
(SEQ ID NO: 14)
QYHISKLSLSAETQDF
wherein said isolated peptide comprises from 16 to 50 amino acids.

30. The peptide according to claim 26, further comprising a peptide tag, a cell penetrating sequence and/or a compound extending half-life of the peptide.

31. The peptide according to claim 30, comprising the amino acid sequence
(SEQ ID NO: 15)
VKKKKIKAEIKISFLRMKNRNSASQVASSL.

32. The peptide according to claim 29, further comprising a peptide tag, a cell penetrating sequence and/or a compound extending half-life of the peptide.

33. The peptide according to claim 32, comprising the amino acid sequence
(SEQ ID NO: 16)
VKKKKIKAEIKIQYHISKLSLSAETQDF.

34. A pharmaceutical composition comprising a peptide according to claim 26.

35. A pharmaceutical composition comprising a peptide according to claim 29.

36. A method for treating cancer in a subject, comprising administering to a subject in need thereof a therapeutically effective amount of a peptide comprising the amino acid sequence SEQ ID NO: 1 or SEQ ID NO:14, or a pharmaceutical composition comprising a peptide comprising the amino acid sequence SEQ ID NO: 1 or SEQ ID NO: 14.

37. A method for treating cancer in a subject, comprising administering to a subject in need thereof a therapeutically effective amount of a peptide comprising the amino acid sequence SEQ ID NO: 1 or SEQ ID NO:14, or a pharmaceutical composition comprising a peptide comprising the amino acid sequence SEQ ID NO: 1 or SEQ ID NO: 14, wherein said peptide is as defined in claim 26.

38. A method for treating cancer in a subject, comprising administering to a subject in need thereof a therapeutically effective amount of a peptide comprising the amino acid sequence SEQ ID NO: 1 or SEQ ID NO:14, or a pharmaceutical composition comprising a peptide comprising the amino acid sequence SEQ ID NO: 1 or SEQ ID NO: 14, wherein said peptide is as defined in claim 29.

39. The method according to claim 36, wherein said cancer is a solid cancer, preferably an epithelial cancer.

40. The method according to claim 36, wherein said cancer is breast, lung, colon pancreas, bladder or head and neck cancer, preferably said cancer is a triple negative breast cancer.

41. The method according to claim 36, wherein said cancer comprises cells with high Kindlin-1 expression level.

42. The method according to claim 36, wherein said cancer is an EGFR/RAS-driven cancer.

43. A polypeptide that comprises a peptide as defined in claim 26, wherein the polypeptide comprises no more than 50 consecutive amino acids of human Kindlin-1.

44. A polypeptide that comprises a peptide as defined in claim 29, wherein the polypeptide comprises no more than 50 consecutive amino acids of human Kindlin-1.

45. A method for preventing and/or treating metastases in a subject having cancer, comprising administering to a subject in need thereof a therapeutically effective amount of a peptide comprising the amino acid sequence SEQ ID NO: 1 or SEQ ID NO: 14, or a pharmaceutical composition comprising a peptide comprising the amino acid sequence SEQ ID NO: 1 or SEQ ID NO: 14.

46. The method A method for treating cancer in a subject, comprising administering to a subject in need thereof a therapeutically effective amount of a peptide comprising the amino acid sequence SEQ ID NO: 1 or SEQ ID NO:14, or a pharmaceutical composition comprising a peptide comprising the amino acid sequence SEQ ID NO: 1 or SEQ ID NO:14, wherein said pharmaceutical composition is as defined in claim 34.

47. A method for treating cancer in a subject, comprising administering to a subject in need thereof a therapeutically effective amount of a peptide comprising the amino acid sequence SEQ ID NO: 1 or SEQ ID NO:14, or a pharmaceutical composition comprising a peptide comprising the amino acid sequence SEQ ID NO: 1 or SEQ ID NO: 14, wherein said pharmaceutical composition is as defined in claim 35.


SEQUENCE LISTING (41 sequences)

SEQ ID NO: 1 - protein, 5 aa, synthetic construct
SFLRM

SEQ ID NO: 2 - protein, 22 aa, synthetic construct
LNILSFLRMKNRNSASQVASSL

SEQ ID NO: 3 - protein, 18 aa, synthetic construct
SFLRMKNRNSASQVASSL

SEQ ID NO: 4 - protein, 15 aa, synthetic construct
SFLRMKNRNSASQVA

SEQ ID NO: 5 - protein, 13 aa, synthetic construct
SFLRMKNRNSASQ

SEQ ID NO: 6 - protein, 12 aa, synthetic construct
ILSFLRMKNRNS

SEQ ID NO: 7 - protein, 12 aa, synthetic construct
LNILSFLRMKNR

SEQ ID NO: 8 - protein, 11 aa, synthetic construct
SFLRMKNRNSA

SEQ ID NO: 9 - protein, 10 aa, synthetic construct
SFLRMKNRNS

SEQ ID NO: 10 - protein, 9 aa, synthetic construct
SFLRMKNRN

SEQ ID NO: 11 - protein, 8 aa, synthetic construct
SFLRMKNR

SEQ ID NO: 12 - protein, 7 aa, synthetic construct
SFLRMKN

SEQ ID NO: 13 - protein, 6 aa, synthetic construct
SFLRMK

SEQ ID NO: 14 - protein, 16 aa, synthetic construct
QYHISKLSLSAETQDF

SEQ ID NO: 15 - protein, 30 aa, synthetic construct
VKKKKIKAEIKISFLRMKNRNSASQVASSL

SEQ ID NO: 16 - protein, 28 aa, synthetic construct
VKKKKIKAEIKIQYHISKLSLSAETQDF

SEQ ID NO: 17 - protein, 12 aa, synthetic construct
VKKKKIKAEIKI

SEQ ID NO: 18 - DNA, 51 bp, synthetic construct
  source 1..51: mol_type = other DNA
catggagattcacaaatatcaaagcggcgaatgtaaactgggaaacccggc

SEQ ID NO: 19 - DNA, 51 bp, synthetic construct
  source 1..51: mol_type = other DNA
gtacctctaagtgtttatagtttcgccgcttacatttgaccctttgggccg

SEQ ID NO: 20 - protein, 24 aa, synthetic construct
VKKKKIKAEIKIILSFLRMKNRNS

SEQ ID NO: 21 - protein, 24 aa, synthetic construct
VKKKKIKAEIKILNILSFLRMKNR

SEQ ID NO: 22 - skipped

SEQ ID NO: 23 - protein, 17 aa, synthetic construct
VKKKKIKAEIKISFLRM

SEQ ID NO: 24 - protein, 18 aa, synthetic construct
VKKKKIKAEIKISFLRMK

SEQ ID NO: 25 - protein, 19 aa, synthetic construct
VKKKKIKAEIKISFLRMKN

SEQ ID NO: 26 - protein, 20 aa, synthetic construct
VKKKKIKAEIKISFLRMKNR

SEQ ID NO: 27 - protein, 21 aa, synthetic construct
VKKKKIKAEIKISFLRMKNRN

SEQ ID NO: 28 - protein, 22 aa, synthetic construct
VKKKKIKAEIKISFLRMKNRNS

SEQ ID NO: 29 - protein, 23 aa, synthetic construct
VKKKKIKAEIKISFLRMKNRNSA

SEQ ID NO: 30 - protein, 25 aa, synthetic construct
VKKKKIKAEIKISFLRMKNRNSASQ

SEQ ID NO: 31 - protein, 27 aa, synthetic construct
VKKKKIKAEIKISFLRMKNRNSASQVA

SEQ ID NO: 32 - protein, 29 aa, synthetic construct
  REGION 1..29: note = Synthetic peptide
VKKKKIKAEIKIFLRMKNRNSASQVASSL

SEQ ID NO: 33 - protein, 28 aa, synthetic construct
  REGION 1..28: note = Synthetic peptide
VKKKKIKAEIKILRMKNRNSASQVASSL

SEQ ID NO: 34 - protein, 26 aa, synthetic construct
  REGION 1..26: note = Synthetic peptide
VKKKKIKAEIKIMKNRNSASQVASSL

SEQ ID NO: 35 - protein, 24 aa, synthetic construct
  REGION 1..24: note = Synthetic peptide
VKKKKIKAEIKINRNSASQVASSL

SEQ ID NO: 36 - protein, 24 aa, synthetic construct
  REGION 1..24: note = Synthetic peptide
VKKKKIKAEIKINSASQVASSLEN

SEQ ID NO: 37 - protein, 30 aa, synthetic construct
  REGION 1..30: note = Synthetic peptide
VKKKKIKAEIKIAFLRAKNRNSASQVASSL

SEQ ID NO: 38 - protein, 30 aa, synthetic construct
  REGION 1..30: note = Synthetic peptide
VKKKKIKAEIKISFLRAKNRNSASQVASSL

SEQ ID NO: 39 - protein, 30 aa, synthetic construct
  REGION 1..30: note = Synthetic peptide
VKKKKIKAEIKIAFLRMKNRNSASQVASSL

SEQ ID NO: 40 - protein, 9 aa, synthetic construct
PRRSFLRMP

SEQ ID NO: 41 - protein, 677 aa, Homo sapiens
MLSSTDFTFASWELVVRVDHPNEEQQKDVTLRVSGDLHVGGVMLKLVEQINISQDWSDFALWWEQKHCWLLKTHWTLDKYGVQADAKLLFTPQHKMLRLRLPNLKMVRLRVSFSAVVFKAVSDICKILNIRRSEELSLLKPSGDYFKKKKKKDKNNKEPIIEDILNLESSPTASGSSVSPGLYSKTMTPIYDPINGTPASSTMTWFSDSPLTEQNCSILAFSQPPQSPEALADMYQPRSLVDKAKLNAGWLDSSRSLMEQGIQEDEQLLLRFKYYSFFDLNPKYDAVRINQLYEQARWAILLEEIDCTEEEMLIFAALQYHISKLSLSAETQDFAGESEVDEIEAALSNLEVTLEGGKADSLLEDITDIPKLADNLKLFRPKKLLPKAFKQYWFIFKDTSIAYFKNKELEQGEPLEKLNLRGCEVVPDVNVAGRKFGIKLLIPVADGMNEMYLRCDHENQYAQWMAACMLASKGKTMADSSYQPEVLNILSFLRMKNRNSASQVASSLENMDMNPECFVSPRCAKRHKSKQLAARILEAHQNVAQMPLVEAKLRFIQAWQSLPEFGLTYYLVRFKGSKKDDILGVSYNRLIKIDAATGIPVTTWRFTNIKQWNVNWETRQVVIEFDQNVFTAFTCLSADCKIVHEYIGGYIFLSTRSKDQNETLDEDLFHKLTGGQD